(19) United States
(12) Patent Application Publication
(10) Pub. No.: US 2009/0112139 A1
(43) Pub. Date: Apr. 30, 2009

(54) Device to elevate to foot

(76) Inventor: Clote Chapman, Jackson, MS (US)

Correspondence Address: THE INVENTORS NETWORK, INC., 332 ACADEMY STREET, CARNEGIE, PA, 15106, US

(21) Appl. No.: 11/978,039

(22) Filed: Oct. 29, 2007

(51) Int. Cl. A61F 5/00

(52) U.S. Cl. 602/23

(57) ABSTRACT

A lightweight portable adjustable foot elevational device for elevating and protecting the foot to alleviate foot ailments and to facilitate surgery recuperation or healing from other injuries includes a foam base having a padded member mounted thereto and upon which the individual's foot is placed with the padded member capable of selective incremental vertical adjustment relative to the stationary base for changing the inclination or elevation of the foot (and lower leg) and a u-shaped padded foot guard pivotally mounted to padded member and capable of being pivoted forward or rearward relative to the seated individual for allowing the individual to first position his/her foot upon the padded member, and then for being pivoted to a position for protecting the individual's foot.

### FIELD OF THE INVENTION

The present invention pertains to medical limb supporting devices, and more particularly pertains to a device that can adjustably elevate the foot of an individual.

### BACKGROUND OF THE INVENTION

Injuries, ailments, and physical problems with the lower extremities often times require a long period of convalescence and special requirements to facilitate the healing process. The above conditions are compounded by the obvious fact that the legs are needed for ambulation, and unless one is completely bedridden or given medical instructions to stay in bed for an extended period of time, it is impossible to provide the lower extremities (lower legs, ankles, and feet) with an uninterrupted period of rest, recuperation, and non-use.

One of the problems with injuries, ailments and recuperation from surgeries to the lower extremities is maintaining the elevation of the foot, for example, to facilitate healing, increase comfort, prevent swelling from the pressure of the foot against a surface whether a floor or bed, and to maintain a wound site relatively clean to minimize infection. The usual way to do this is to prop the foot up on a pillow or pillows with the height of the elevation determined by the number of pillows stacked on atop the other. However, this is an inadequate expedient as pillows are bulky and the elevational adjustment is imprecise and ad hoc. Moreover, it is also awkward to carry around a stack of pillows if one wants to move from the bedroom to the living room. A more compact and portable device is needed that allows for some adjustment in the elevation and inclination of the lower extremities, isn't nearly as bulky as pillows stacked on top of each other, and can be used on all surfaces. It would b preferable if one didn't have to use pillows or blankets tightly folded or rolled together for outdoor recuperation and injury recovery. Thus, the prior art discloses a range of devices for treating and alleviating injuries, ailments, and physical conditions to the lower extremities through the elevation of the lower extremities.

For example, the Drennan patent (U.S. Pat. No. 5,449,339) discloses a heel supporting protective boot that includes a flexible body into which the foot is placed for enclosure therein and to which a foam material can be adhesively secured for preventing external rotation of the foot.

The Shepich patent (U.S. Pat. No. 5,603,336) discloses a bed footbox for medical patient's that include a pair of receptacles, one for each foot, interconnected by a cushioned pad that extends across the front of the receptacles.

The Ewing patent (U.S. Pat. No. 5,765,564) discloses a medical limb support assembly that includes an inflatable tubular bladder attached to the foot of the bed and extending transverse thereto for supporting the feet of the individual upon the bladder.

The Klein patent (U.S. Pat. No. 5,957,874) discloses a heel-elevating device that includes a u-shaped leg support having spaced leg extensions and a u-shaped comfort liner placed contiguous within and to the leg extensions for supporting the foot and heel of the individual placed therein.

The Jackson patent (U.S. Pat. No. 6,175,979 B1) discloses an inflatable orthopedic pillow having a size and shape for effectively elevating and maintaining the lower extremities of the individual.

The Klein patent (U.S. Pat. No. 6,572,573 B1) discloses a heel elevating device that includes a u-shaped lower leg support, spaced upright leg extensions, and a comfort liner for supporting the foot, heel, and ankle of the individual within and between the leg extensions.

Nonetheless, despite the ingenuity of the above devices, there remains a need for a foot elevation device whose height is adjustable to accommodate the particular needs of each individual user of the device.

### SUMMARY OF THE INVENTION

The present invention comprehends a device to elevate the foot of an individual, and especially the foot of the individual that has sustained an injury or has had surgery performed thereon. The device of the present invention is also useful in alleviating various foot ailments and problems.

The device to elevate the foot of the individual, which is also adjustable in height, can be referred to as an adjustable foot elevation device for raising and inclining the foot, and, by extension, the lower leg relative to the thigh and upper leg of the individual. The foot and lower leg would not necessarily be raised higher than the torso or head of the individual as the individual may be sitting in a chair, sitting in bed, or sitting on the floor when using this device.

The adjustable foot elevation device includes a generally square-shaped foam base. The foam base has a bottom or underside that contacts the floor surface. Disposed upon the foam base and substantially covering the base is a soft padded member. The soft padded member is encased in a covering. In addition, the soft padded member has a substantially rigid bottom or floor support. Pivotally mounted to two opposed sides of the padded member is a u-shaped padded foot guard. The foot guard can pivot toward (fore) the individual or away (aft) from the individual to allow the individual to slip his or her foot between the padded member and the horizontal cross portion of the padded member.

The device to elevate the foot includes a height adjustment means that, in the preferred embodiment, provides for an elevational adjustment of the padded member relative to the foam base of between one and six inches. The adjustment means includes a manually operable rotatable adjustment button mounted on one spindle that interconnects the lower attachment portion of one leg portion of the foot guard to the padded member. The adjustment button is interconnected to any conventional mechanism, such as a rack and pinion arrangement, for selectively raising or lowering the padded member relative to the foam base. A clearance area or space is created between the support floor of the padded member and the foam base as the padded member is raised relative to the stationary foam base.

It is an objective of the present invention to provide an adjustable foot elevation device that that alleviates foot problems or injuries by protecting and elevating the foot for better healing.

It is another objective of the present invention to provide an adjustable foot elevation device that increases the comfort of the elevated foot by keeping bedding off the foot.

It is yet another objective of the present invention to provide an adjustable foot elevation device that maintains the elevation of the foot thereby reducing the amount of swelling and pain that may occur.

It is still yet another objective of the present invention to provide an adjustable foot elevation device that is ideal for individuals suffering from common foot ailments and for individuals recovering from surgery to the foot and lower leg.

Still yet another objective of the present invention is to provide an adjustable foot elevation device that is lightweight, portable, and easily positionable on a bed, couch, or floor for providing the individual with maximum comfort and support.

These and other objects, features, and advantages will become apparent to those skilled in the art upon a perusal of the following detailed description read in conjunction with the accompanying drawing figures and appended claims.

### BRIEF DESCRIPTION OF THE DRAWINGS

FIG. 1 is perspective view of the adjustable foot elevation device of the present invention;

FIG. 2 is a side elevational view of the adjustable foot elevation device of the present invention illustrating the disposition and placement of the foot of the individual on the upper surface of the padded member;

FIG. 3 is a side elevational view of the adjustable foot elevation device of the present invention illustrating the rotation of the padded foot guard fore and aft relative to the foot and lower leg of the individual;

FIG. 4 is a side elevational view of the adjustable foot elevation device of the present invention illustrating the vertical adjustment of the padded member to raise and lower the foot;

FIG. 5 is a top plan view of the adjustable foot elevation device of the present invention illustrating the disposition of the foot of the individual relative to the padded foot guard and the padded member;

FIG. 6 is a perspective view of the adjustable foot elevation device of the present illustrating the displacement of the padded member relative to the raising and lowering of the padded member; and

FIG. 7 is a top plan view of the adjustable foot elevation device of the present invention illustrating the mechanism that facilitates the raising and lowering of the padded member.

### DETAILED DESCRIPTION OF THE PREFERRED EMBODIMENT

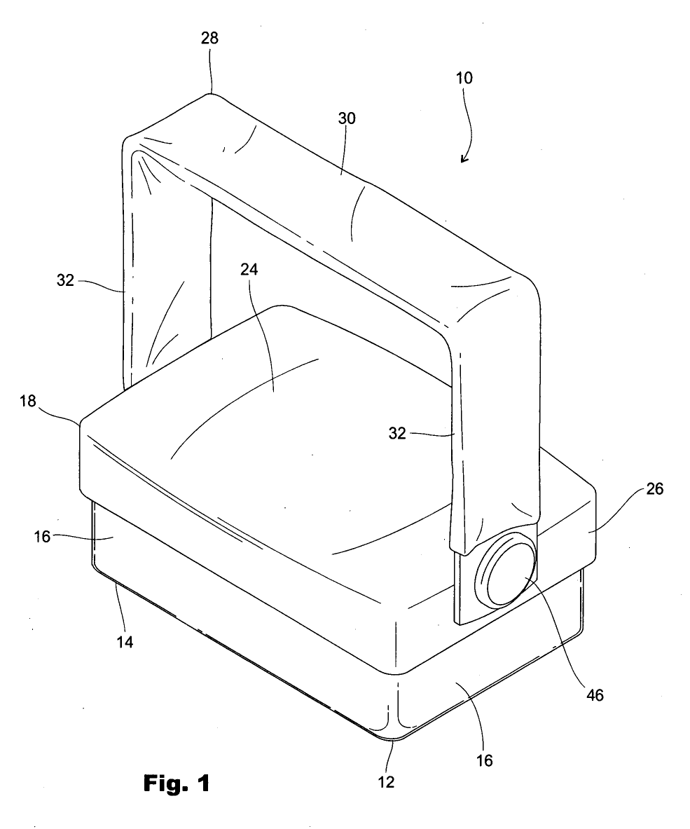

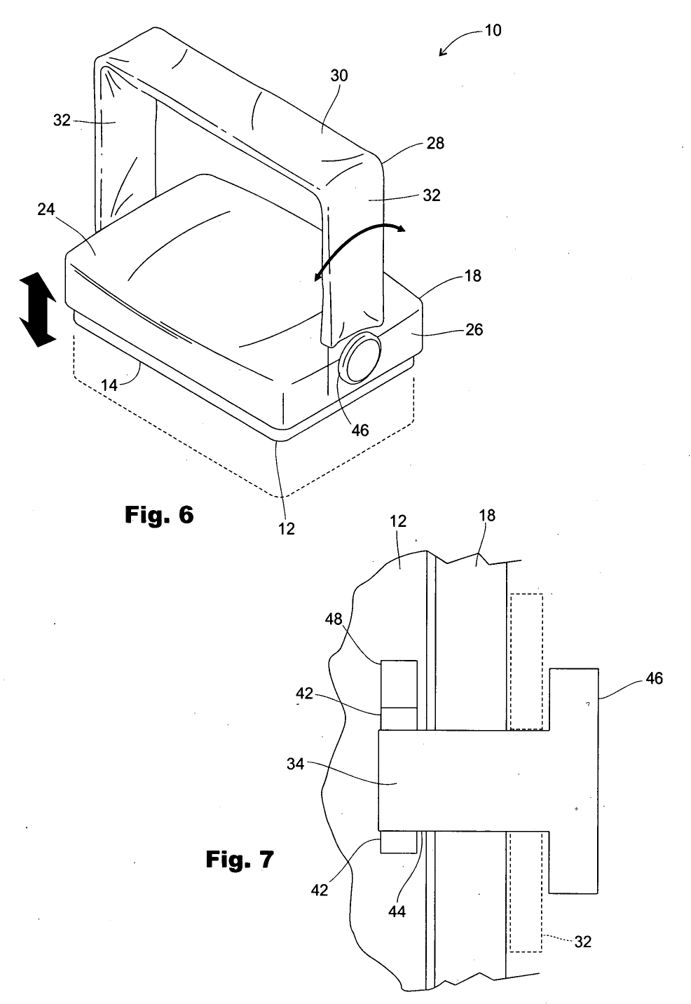

Illustrated in FIGS. 1-7 is a lightweight portable device to support, protect and elevate the foot, and by extension, the lower leg for a number of health and recuperative reasons that can include: elevating the foot as part of the process of healing and recovery after foot surgery: elevating and inclining the foot to relieve pressure and minimize infection to wound and surgical sites; and elevating and inclining the foot for relaxing and sleeping in comfort as part of the process of convalescing or alleviating a long-term or congenital ailment or condition involving the foot and/or lower leg.

Thus, illustrated in FIGS. 1-7 is an adjustable foot elevation device 10 that includes a generally rectangular-shaped base 12 preferably composed of foam or a foam-type material and including a flat bottom surface or underside 14 an upper inner flat surface and four continuously adjoined base sidewalls 16. Mounted to and upon the foam base 12 is a rectangular-shaped padded member 18 having slightly larger length and width dimensions than the foam base 12. In effect, the padded member 18 surrounds and partially overhangs about the foam base 12. The padded member 18 includes a covering 20 that encloses or encases therein a soft padding 21. Extending across the interior of the padded member 18 is a rigid floor support 22. An upper foot supporting surface 24 and depending and adjoined sidewalls 26 further define the padded member 18.

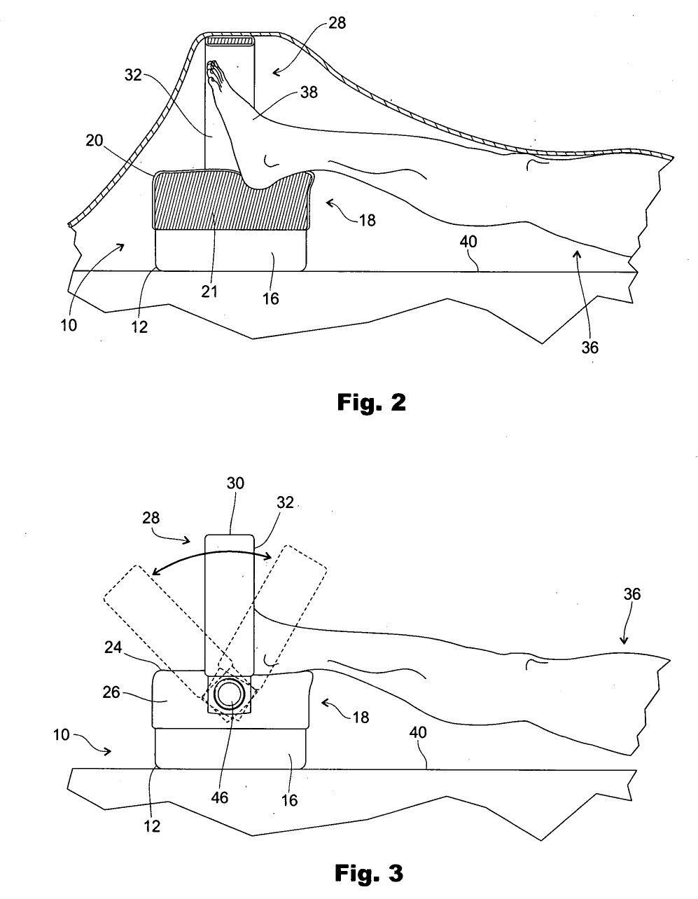

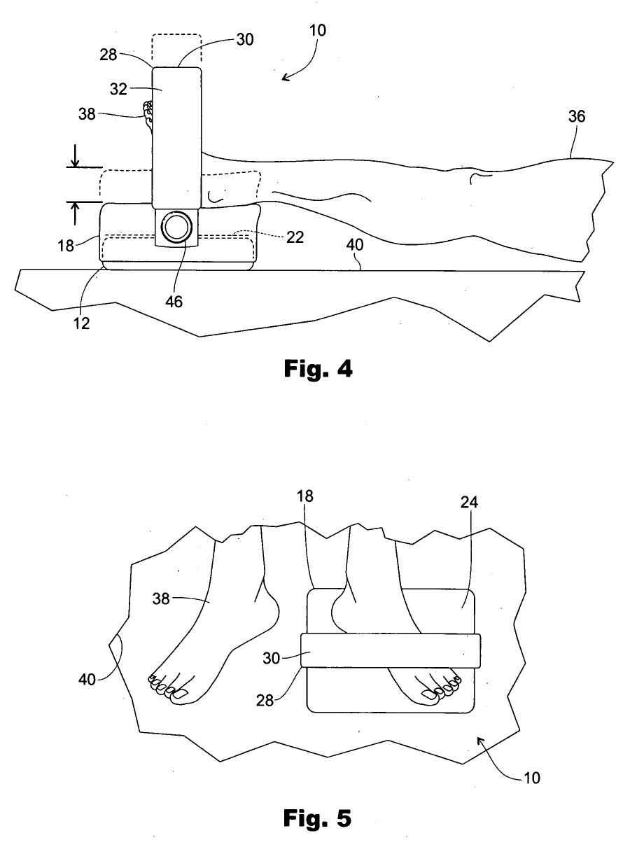

As shown in FIGS. 1-7, the portable and adjustable foot elevation device 10 also includes a u-shaped padded foot guard 28 that is pivotally mounted to two opposed sidewalls 26 of the padded member 18. The padded foot guard 28 includes a horizontal cross portion 30 and two opposed leg portions 32 integrally attached to the horizontal cross portion 30. Mounted to and slightly projecting outwardly from each opposed sidewall 26 is a spindle or axle 34, and each opposed leg portion 32 is pivotally mounted to each respective spindle 34 at the lowermost ends of the leg portions 32. This allows the padded foot guard 28 to pivot toward (fore) the seated, recumbent or resting individual 36, and also to pivot away (aft) from the seated, recumbent or resting individual 36. The padded foot guard 28 is pivotable toward or away from the individual 36, as shown in FIG. 3, so that the individual's foot 38 can be placed upon the upper foot supporting surface 24 of the padded member 18. After the foot 38 is properly positioned the foot guard 28 is pivoted to a generally upright vertical position over and above the foot 38 to protect the foot 38 as shown in FIGS. 2 and 4. It should also be noted that the padded member 18 could be used as a handle to pick up and move or transport the device 10 as needed. Moreover, FIG. 2 shows the padded foot guard 28 in the vertically upright position for keeping a covering, such as a blanket or sheet, off the individual's foot 38 thus keeping the foot 38 free from rubbing, chafing, discomfort, or other encumbrance caused by the covering rubbing against or laying upon the foot 38 or lower leg.

The foot elevational device 10 also includes an incremental height adjustment means to vary the height the foot 38 is elevated or raised above the supporting surface, such as the representative living room floor 40 shown in FIGS. 2-5. The incremental height adjustment means allows the individual to manually adjust and vary the change in foot elevation. The incremental height adjustment means of the present invention includes a pinion gear 42 mounted to the inner end 44 of one spindle 34. A manual and rotatable adjustment button or knob 46 is exteriorly mounted to the spindle 34, and the button 46 and spindle 34 can be an integrally formed as a one-piece unit as shown in FIG. 7. The rotatable mounting of the lower end of one leg portion 32 of the padded member 18 to the spindle 34 immediately inboard of the adjustment button 46 is also shown in FIG. 7. Fixedly mounted to the foam base 12 and extending upwardly therefrom for engagement to the pinion gear 42 that is located on the inner end 44 of the spindle 34 is a rack 48. The rack 48 is stationarily mounted to the foam base 12 adjacent an edge or periphery of the base 12. When the individual desires to change the height of the device 10, he or she simply rotates the adjustment button 46 clockwise or counterclockwise, and this rotation causes the pinion gear 42 to engage the rack 48 thereby resulting in the upward or downward movement and displacement the padded member 18 relative to the supporting surface, such as floor 40, and the base 12. In the preferred embodiment of the invention, the range of elevational change is between one and six inches; and this range of adjustment for elevating the foot 38 is shown in FIGS. 2-4.

The foregoing detailed description is to be clearly understood as given by way of illustration and example only and numerous modifications, alterations, or variations are possible and practicable to those skilled in the art while yet remaining within the spirit of the invention and the scope of the appended claims hereof.

I claim:

1. A device for supporting and elevating the foot, comprising:
a base composed of foam material;
a padded member mounted upon the base and capable of selective upward or downward movement relative to the base;
the padded member including an upper foot supporting surface;
a padded foot guard pivotally mounted to the padded member;
an adjustment button mounted to the padded member for raising and lowering the padded member; and
incremental height adjustment means interconnecting the padded member to the base so that rotation of the adjustment button clockwise and counterclockwise raises and lowers the padded member thereby changing the elevation of the foot.

2. The device for supporting and elevating the foot of claim 1 wherein the padded foot guard includes a horizontal cross portion and a pair of opposed leg portions.

3. The device for supporting and elevating the foot of claim 2 wherein the padded member includes an upper foot supporting surface and a plurality of depending and adjoined sidewalls.

4. The device for supporting and elevating the foot of claim 3 wherein the padded foot guard pivots to allow for the positioning and placement of the foot of the individual upon the upper foot supporting surface of the padded member.

5. The device for supporting and elevating the foot of claim 4 wherein the range of elevational change and adjustment of the padded member with respect to the base is from between one and six inches.

6. A device for supporting and elevating the foot, comprising:
a base composed of foam material;
the base including a flat bottom surface and four continuously adjoined base sidewalls;
a padded member mounted upon the base and capable of selective upward and downward movement relative to the base;
the padded member including an upper foot supporting surface;
a u-shaped padded foot guard pivotally mounted to the padded member;
the padded foot guard including a horizontal cross portion and a pair of oppositely disposed leg portions integrally attached to the horizontal cross portion;
an adjustment button mounted to the padded member for raising and lowering the padded member; and
incremental height adjustment means interconnecting the padded member to the base so that rotation of the adjustment button clockwise and counterclockwise initiates the raising and lowering of the padded member and thereby changing the elevation of the foot.

7. The device for supporting and elevating the foot of claim 6 wherein the padded foot guard pivots to allow for the positioning and placement of the foot of the individual upon the upper foot supporting surface of the padded member.

8. The device for supporting and elevating the foot of claim 7 wherein the range of elevational change and adjustment of the padded member with respect to the base is at least between one and six inches.
